(19) United States
(12) Patent Application Publication
(10) Pub. No.: US 2013/0171083 A1
(43) Pub. Date: Jul. 4, 2013

(54) PHOTO-CURABLE COSMETIC COMPOSITIONS

(71) Applicants: Chunhua LI, Scotch Plains, NJ (US); Hy Si BUI, Piscataway, NJ (US); Jean-Thierry SIMONNET, Mamaroneck, NY (US); Xian Zhi ZHOU, Leonia, NJ (US); Nicholas Stergios LIOUTAS, Philadelphia, PA (US)

(72) Inventors: Chunhua LI, Scotch Plains, NJ (US); Hy Si BUI, Piscataway, NJ (US); Jean-Thierry SIMONNET, Mamaroneck, NY (US); Xian Zhi ZHOU, Leonia, NJ (US); Nicholas Stergios LIOUTAS, Philadelphia, PA (US)

(73) Assignee: L'OREAL S.A., Paris (FR)

(21) Appl. No.: 13/341,875

(22) Filed: Dec. 30, 2011

(51) Int. Cl. A61K 8/81; A61Q 3/02

(52) U.S. Cl. 424/61

(57) ABSTRACT

Disclosed herein are cosmetic compositions comprising (1) at least one polyvinyl alcohol-styrylpyridinium polymer and (2) at least one latex film-forming polymer. Also disclosed herein are methods for making up and/or enhancing the appearance of a keratinous substrate comprising (1) forming a film on the keratinous substrate by applying to said keratinous substrate a cosmetic composition comprising (a) at least one polyvinyl alcohol-styrylpyridinium polymer and (2) at least one latex film-forming polymer and (2) exposing the film to radiation chosen from UV and visible light radiation.

### FIELD OF THE DISCLOSURE

The disclosure relates to cosmetic compositions comprising (1) at least one polyvinyl alcohol-styrylpyridinium polymer and (2) at least one latex film-forming polymer. Latex film formers useful in various embodiments of the disclosure may be chosen from acrylate copolymers, styrene/acrylate copolymers, acrylamide/acrylate copolymers, polyurethanes, and derivatives thereof. Compositions according to various embodiments of the disclosure may exhibit improved properties, such as improved water- and/or oil- and/or chemical-resistance, shine, adhesion, strength, and/or long wear.

### BACKGROUND

Gel-based cosmetic compositions are known. For example, gel-based nail polishes have become increasingly popular in recent years, as they may provide improved properties over conventional nail polishes, such as extended wear and improved shine. However, consumers have raised safety concerns regarding the small molecules, such as the presence of photoinitiators and monomers in available gel-based nail polishes. In addition, gel-based nail polishes must be cured using UV radiation and can be difficult to remove. Furthermore, the application of gel-based nail polishes is expensive, time-consuming, and requires salon services for application and removal.

Thus, there is a desire in the cosmetic industry to provide consumers with safer and/or more convenient photo-curable cosmetic products that do not comprise small molecules such as photoinitiators, do not require curing with UV radiation, and/or exhibit improved ease of application and/or removal. In addition, it is desired that such photo-curable cosmetic products exhibit improved properties such as improved water- and/or oil- and/or chemical-resistance, shine, adhesion, strength, and long wear. As such, there is a continuous need to invent novel cosmetic compositions and methods of making up keratinous fibers which demonstrate one or more of said improved properties.

### DESCRIPTION OF EXEMPLARY EMBODIMENTS

The disclosure relates, in various embodiments, to cosmetic compositions comprising (1) at least one polyvinyl alcohol-styrylpyridinium polymer and (2) at least one latex film former.

Polyvinyl alcohol-styrylpyridinium (“PVA-SbQ”) polymers have been widely used in various arts as photocrosslinkable materials due to their high photosensitivity and good storage stability. Upon exposure to UV or visible light radiation, PVA-SbQ polymers are capable of crosslinking via a 2+2 cycloaddition reaction without the use of a photoinitiator. However, the use of PVA-SbQ polymers in the cosmetic arts has been limited due to their highly hydrophilic nature. Thus, known cosmetic compositions comprising PVA-SbQ polymers may have less than satisfactory properties. For example, nail varnish compositions comprising PVA-SbQ may exhibit poor adhesion to the nail and/or may not be sufficiently water- and/or oil-resistant.

It has now been surprisingly discovered, however, that by incorporating (1) at least one polyvinyl alcohol-styrylpyridinium polymer and (2) at least one latex film former into cosmetic compositions, one or more cosmetic properties such as improved water- and/or oil- and/or chemical-resistance, shine, adhesion, strength, and/or long wear can be improved.

According to the disclosure, PVA-SbQ polymers useful in various embodiments may be chosen from polyvinyl alcohol polymers comprising at least one pendent styrylpyridinium group of formula (I):

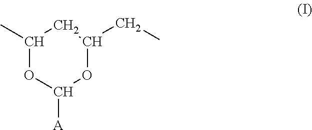

wherein A is chosen from:

(1) groups of formula (II):

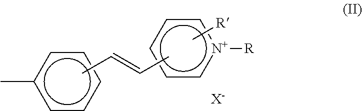

wherein R is chosen from hydrogen, alkyl groups, and hydroxyalkyl groups; R′ is chosen from hydrogen and alkyl groups; and X⁻ is an anion chosen from chloride, bromide, iodide, sulfates, methosulfates, perchlorates, phosphates, and sulfonates; and

(2) groups of formula (III):

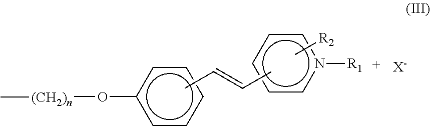

wherein R₁ is chosen from hydrogen, alkyl groups, and aralkyl groups; R₂ is chosen from hydrogen and alkyl groups; X⁻ is an anion chosen from chloride, bromide, iodide, sulfates, methosulfates, perchlorates, phosphates, and sulfonates; and n is an integer ranging from 1 to 6.

By way of non-limiting example, PVA-SbQ polymers include those described in U.S. Pat. Nos. 4,272,620, 4,287,335, 4,339,524, 4,564,580, and 4,777,114, the disclosures of which are incorporated herein by reference in their entireties. For example, the at least one PVA-SbQ polymer may be chosen from polyvinyl alcohol polymers comprising at least one pendent styrylpyridinium group chosen from:
1-methyl-2-{m-(2,2-dimethoxyethoxy)-styryl}-pyridinium iodide,
1-methyl-2-{o-(2,2-dimethoxyethoxy)-styryl}-pyridinium iodide,
1-methyl-4-{p-(2,2-dimethoxyethoxy)-styryl}-pyridinium p-toluenesulfonate,
1-methyl-4-{p-(2,2-dimethoxyethoxy)-styryl}-pyridinium methosulfate,
1-methyl-2-(m-formylstyryl)-pyridinium p-toluenesulfonate,
1-methyl-2-(p-formylstyryl)-pyridinium p-toluenesulfonate,
1-methyl-2-(p-formylstyryl)-pyridinium iodide,
1-methyl-2-(p-formylstyryl)-pyridinium methosulfate,
1-methyl-4-(p-formylstyryl)-pyridinium p-toluenesulfonate,
1-methyl-4-(p-formylstyryl)-pyridinium methosulfate,
1-(2-hydroxyethyl)-2-(p-formylstyryl)-pyridinium chloride, and
1-carbamoylmethyl-4-(p-formylstyryl)-pyridinium chloride.

According to various embodiments of the disclosure, the at least one PVA-SbQ polymer may be chosen from poly(vinyl alcohol), N-methyl-4(4′-formylstyryl)pyridinium methosulfate acetal. Exemplary commercial PVA-SbQ polymer products that may be used in accordance with the disclosure include, but are not limited to, the product sold by Polysciences, Inc. under catalogue number 22570-75; and products sold by Murakami Co. Ltd. under the names EMULSION AD-V (poly(vinyl alcohol) with pendent pyridinium (and) ethylene/VA copolymer), EMULSION AD-V2 (poly(vinyl alcohol) with pendent pyridinium (and) ethylene/VA copolymer), and EMULSION AD-V2 PVA-SBQ (poly(vinyl alcohol) with pendent pyridinium).

In at least one exemplary embodiment, the molar ratio of PVA:SbQ in the at least one PVA-SbQ polymer may range from about 99.5:0.5 to about 85:15, such as, for example, about 99:1 to about 90:10, about 98:2 to about 92:8, or about 97:3 to about 95:5. According to another exemplary embodiment of the disclosure, the at least one PVA-SbQ polymer may have a molecular weight ranging from about 30,000 to about 100,000, such as, for example, about 40,000 to about 80,000, or about 50,000 to about 70,000. In yet another exemplary embodiment, the at least one PVA-SbQ may have a degree of polymerization ranging from about 100 to about 2500, for instance, from about 200 to about 2000, or from about 300 to about 1800, or from about 500 to 1000. In various exemplary embodiments, the at least one PVA-SbQ may have a degree of polymerization ranging from about 200 to about 800, for example, from about 300 to about 500.

According to at least certain embodiments of the disclosure, the at least one PVA-SbQ polymer is capable of crosslinking without the requirement of a photoinitiator. For example, without wishing to be bound, it is believed to be capable of crosslinking via a 2+2 cycloaddition reaction, upon exposure to UV or visible light radiation. For example, the at least one PVA-SbQ is capable of crosslinking without a photoinitiator upon exposure to radiation having a wavelength ranging from about 200 nm to about 800 nm, such as from about 250 nm to about 450 nm, or from about 265 nm to about 350 nm. In one exemplary embodiment, the at least one PVA-SbQ polymer is able to crosslink upon exposure to UV light radiation having a wavelength ranging from about 200 nm to about 400 nm. According to another exemplary embodiment, the at least one PVA-SbQ polymer is able to crosslink upon exposure to visible light radiation having a wavelength ranging from about 400 nm to about 800 nm.

In at least one exemplary embodiment, the at least one PVA-SbQ polymer may be present in the cosmetic composition in an amount ranging from about 0.1% to about 40% by weight, such as from about 0.5% to about 30%, or from about 1% to about 20%. According to one non-limiting embodiment, the at least one PVA-SbQ polymer may be present in the cosmetic composition in an amount ranging from about 5% to about 10% by weight.

As described herein, the cosmetic compositions comprising at least one PVA-SbQ polymer also comprise at least one latex film former. As used herein, a “film former” is understood to mean a polymer capable of forming, alone or in the presence of at least one additional agent which is able to form a film, an isolable film, for instance, a continuous and adherent film, on a keratinous substrate. Examples of suitable film formers include, but are not limited to, polyurethanes, acrylic polymers, vinyl polymers, silicone polymers, and derivatives thereof.

Latex film formers useful according to various embodiments of the disclosure may be chosen from, for example, acrylate copolymers, styrene/acrylate copolymers, acrylamide/acrylate copolymers, polyurethanes, and derivatives thereof. According to various embodiments of the disclosure, the at least one latex film former may be chosen from those having a glass transition temperature (Tg) ranging from about −15° C. to about 90° C., such as from about 0° C. to about 50° C.

In accordance with various exemplary embodiments, acrylate copolymers may be chosen from copolymers comprising two or more monomers chosen from acrylic acid, methacrylic acid, and their simple esters, for example, lower alkyl esters such as methyl, ethyl, and ethylhexyl esters. By way of non-limiting example only, acrylate copolymers may be chosen from ammonium acrylates copolymers, ethyl acrylates copolymers, acrylates/ethylhexylacrylate copolymers, acrylates/octylacrylates copolymers, akly (meth)acrylates copolymers, acrylates/C₁₂-C₂₂ alkylmethacrylate copolymers, ethylacrylate/methacrylic acid copolymer, and t-butyl acrylate/ethyl acrylate/methacrylic acid copolymer. Exemplary commercial acrylate copolymers include, but are not limited to, ALLIANZ™ OPT sold by Ashland Specialty Ingredients; COVACRYL A15 and COVACRYL E14 sold by Sensient Cosmetic Technologies LCW; DAITOSOL 4000 SJT, DAITOSOL 5000 AD, DAITOSOL 5000 SJ, KOBOGUARD® 50A, and KOBOGUARD® 50N sold by Kobo Products, Inc.; DERMACRYL® AQF, YODOSOL 32A707, YODOSOL GH15, YODOSOL GH32, YODOSOL GH33, YODOSOL GH34, YODOSOL GH35, YODOSOL GH800, and YODOSOL GH810 sold by AkzoNobel; LUVIFLEX® SOFT, LUVIMER® 36D, and LUVIMER® 100P sold by BASF; and NEOCRYL XK-90 sold by Neoresins, Inc.

According to further exemplary embodiments of the disclosure, styrene/acrylate copolymers may be chosen from styrene/acrylates/ammonium methacrylate copolymers, such as SYNTRAN® 5760 sold by Interpolymer Corporation. Other non-limiting examples of commercially available styrene/acrylate copolymers include, but are not limited to, DAITOSOL 5000 STY sold by Kobo Products, Inc.; JONCRYL® 77 sold by BASF; NEOCRYL BT-62 sold by Neoresins, Inc.; RHOPLEX™ P-376 and UCAR™ DL 432S sold by Dow Chemical Company; and YODOSOL GH41 and YODOSOL GH840 sold by AkzoNobel.

In further exemplary embodiments, acrylamide/acrylate copolymers may be chosen from acrylic acid/ethyl acrylate/t-butyl acrylamide copolymer, acrylates/octylacrylamide copolymer, and octylacrylamide/acrylates/methacrylates copolymer. Exemplary commercial acrylamide/acrylate copolymers include, but are not limited to AMPHOMER® LV-71 and DERMACRYL® 79 sold by AkzoNobel and ULTRAHOLD® STRONG sold by BASF.

According to additional exemplary embodiments, polyurethanes may be chosen from polyurethane-32, polyurethane-34, and polyurethane-35. Exemplary commercial polyurethanes include, but are not limited to, products sold by Bayer under the trade name BAYCUSAN®, such as BAYCUSAN® C1000, BAYCUSAN® C1001, BAYCUSAN® C1003, and BAYCUSAN® C1004.

In accordance with various exemplary embodiments, the at least one latex film former may be present in the cosmetic composition in an amount ranging from about 10% to about 99% by weight, such as from about 15% to about 90%, or from about 20% to about 80%.

One exemplary embodiment of the disclosure relates to cosmetic compositions comprising (1) at least one polyvinyl alcohol-styrylpyridinium polymer and (2) at least one latex film former chosen from acrylate copolymers, styrene/acrylate copolymers, acrylamide/acrylate copolymers, polyurethanes, and derivatives thereof.

Another exemplary embodiment of the disclosure relates to cosmetic compositions comprising (1) at least one polyvinyl alcohol-styrylpyridinium polymer and (2) at least one latex film former chosen from acrylate copolymers and derivatives thereof, for example, ammonium acrylates copolymers such as DERMACRYL® AQF.

Yet another exemplary embodiment of the disclosure relates to cosmetic compositions comprising (1) at least one polyvinyl alcohol-styrylpyridinium polymer and (2) at least one latex film former chosen from styrene/acrylate copolymers and derivatives thereof, for example, styrene/acrylates copolymers such as JONCRYL® 77 and RHOPLEX™ P-376 and styrene/acrylates/ammonium methacrylates copolymers such as SYNTRAN® 5760.

A further exemplary embodiment of the disclosure relates to cosmetic compositions comprising (1) at least one polyvinyl alcohol-styrylpyridinium polymer and (2) at least one latex film former chosen from polyurethanes and derivatives thereof, for example, polyruethanes such as BAYCUSAN® C1000, BAYCUSAN® C1001, BAYCUSAN® C1003, and BAYCUSAN® C1004.

Also disclosed herein are methods for making up and/or enhancing the appearance of a keratinous substrate comprising (1) forming a film on said keratinous substrate by applying to the keratinous substrate a cosmetic composition comprising (a) at least one polyvinyl alcohol-styrylpyridinium polymer and (b) at least one latex film former, and (2) exposing the film to radiation chosen from UV and visible light radiation.

According to various methods disclosed herein, the film formed on the keratinous substrate may subsequently be exposed to radiation, e.g. chosen from UV and visible light radiation. In at least one exemplary embodiment, the film is exposed to radiation having a wavelength ranging from 200 nm to 800 nm. By way of non-limiting example, the film may be exposed to UV radiation having a wavelength ranging from about 200 nm to about 400 nm and/or visible light radiation having a wavelength ranging from about 400 nm to about 800 nm.

In a further non-limiting embodiment, the film may be exposed to radiation for a time period ranging from about 0.5 minutes to about 60 minutes, for example, from about 1 minute to about 30 minutes, or from about 2 minutes to about 10 minutes.

In addition, other cosmetic ingredients may be included in the cosmetic compositions according to the disclosure. Such ingredients are known, and include but are not limited to solvents (including water), colorants, humectants, emulsifiers, surfactants, preservatives, fragrances, thickeners or texturizers, emollients, and additional film-formers, coalescents, and/or plasticizers. One of skill in the art will be able to select appropriate types and amounts of additional cosmetic ingredients, based on, for example, the type of cosmetic composition being formulated and the desired properties thereof. By way of example only, such additional cosmetic ingredients may be present in the compositions according to the disclosure in a combined amount ranging from about 10% to about 80% by weight, such as from about 15% to about 60%, from about 25% to about 40%, or from about 30% to about 35%.

Exemplary cosmetic compositions contemplated according to the disclosure include compositions intended for application to keratinous substrates, such as the hair, skin, and nails. Such compositions include, but are not limited to, nail compositions (e.g. nail enamel), mascara compositions, make-up compositions (e.g. foundations), sunscreen compositions, and hair-care compositions (e.g. hair-styling compositions). In at least one exemplary and non-limiting embodiment, the cosmetic composition is a nail composition.

Without wishing to be bound by theory, it is believed that by combining the at least one PVA-SbQ polymer with at least one latex film former, the hydrophilic effect of the hydroxyl groups in the PVA-SbQ polymer is reduced, resulting in a cosmetic composition with improved water resistance. It is further believed that the combination of at least one PVA-SbQ polymer and at least one latex film former shows surprising and unexpected effects, imparting improved properties such as, for example, improved water- and/or oil- and/or chemical-resistance, shine, adhesion, strength, and/or long wear. It is further believed that, due to the hydrophilic nature of the PVA-SbQ polymer, cosmetic compositions in accordance with the disclosure will be easier to remove with conventional solvents. It should be noted, however, that compositions according to the disclosure may not impart one or more of the above-referenced improved properties, yet such compositions are intended to be within the scope of the disclosure.

It is to be understood that both the foregoing description and the following Examples are exemplary and explanatory only, and are not to be interpreted as restrictive of the disclosure. Moreover, it should be understood that various features and/or characteristics of differing embodiments herein may be combined with one another. It is therefore to be understood that numerous modifications may be made to the illustrative embodiments and that other arrangements may be devised without departing from the scope of the invention. Other embodiments will be apparent to those skilled in the art from consideration of the disclosure and practice of the various exemplary embodiments disclosed herein.

It is also to be understood that, as used herein the terms “the,” “a,” or “an,” mean “at least one,” and should not be limited to “only one” unless explicitly indicated to the contrary. Thus, for example, the use of “a PVA-SbQ polymer” is intended to mean at least one PVA-SbQ polymer.

Unless otherwise indicated, all numbers used in the specification and claims are to be understood as being modified in all instances by the term “about,” whether or not so stated. It should also be understood that the precise numerical values used in the specification and claims form additional embodiments of the invention, and are intended to include any ranges which can be narrowed to any two end points disclosed within the exemplary ranges and values provided. Efforts have been made to ensure the accuracy of the numerical values disclosed herein. Any measured numerical value, however, can inherently contain certain errors resulting from the standard deviation found in its respective measuring technique.

### EXAMPLES

The following Examples are intended to be non-restrictive and explanatory only, with the scope of the invention being defined by the claims.

### Example 1

### Clear Nail Enamel

A simple clear nail enamel composition was prepared by mixing, using low mechanical shearing force, the components listed in the following Table I.

TABLE I
Preparation of Clear Nail Enamel

| COMPONENT | | |
|---|---|---|
| Trade Name | INCI | WEIGHT % |
| Polyscience 22570-75 | PVA-SbQ Polymer | 9.09 |
| (13.3 wt % active | | |
| ingredient in water) | | |
| DERMACRYL ® AQF | Ammonium Acrylates | 90.91 |
| (45 wt % active ingredient | Copolymer | |
| in water) | | |

### Example 2

### Red Nail Enamel

Three simple red nail enamel compositions were prepared by mixing, using low mechanical shearing force, the components listed in the following Table II. Comparative composition 1, not in accordance with the disclosure, comprises at least one latex film former, but does not comprise at least one PVA-SbQ polymer. Comparative composition 2, not in accordance with the disclosure, comprises at least one PVA-SbQ polymer, but does not comprise at least one latex film former. Inventive composition 3, in accordance with the disclosure, comprises at least one latex film former and at least one PVA-SbQ polymer.

TABLE II
Preparation of Red Nail Enamel

| | | WEIGHT % | | |
|---|---|---|---|---|
| | | Compo- | Compo- | Compo- |
| | | sition 1 | sition 2 | sition 3 |
| COMPONENT | | (compar- | (compar- | (inven- |
| Trade Name | INCI | ative) | ative) | tive) |
| PS22570 | PVA-SbQ | | 95.5 | 9.5 |
| (13.3 wt % | Polymer | | | |
| in water) | | | | |
| JONCRYL ® 77 | Styrene/Acrylates | 95.5 | | 86 |
| (45.5 wt % | Copolymer | | | |
| in water) | | | | |
| | Mica/pigment | 4.5 | 4.5 | 4.5 |
| TOTAL | | 100 | 100 | 100 |

Each of compositions 1-3 was applied to human nails and evaluated for color uniformity, shine, shine retention, long wear, and ease of removal. These observations are set forth in the following Table III.

Color uniformity was evaluated visually upon application and rated on a scale from 0 to 5, where 0 represents poor color uniformity and 5 represents good color uniformity. Shine was evaluated visually upon application and rated on a scale from 0 to 5, where 0 represents no shine and 5 represents high shine. Shine retention was evaluated visually after a period of about four days and rated on a scale from 0 to 5, where 0 represents no shine retention and 5 represents high shine retention. Wear was evaluated visually over a period of about three hours to about four days after application and rated on a scale from 0 to 5, where 0 represents no adhesion to the nail surface and 5 represents high adhesion. Ease of removal was evaluated visually and rated on a scale from 0 to 5, where 0 represents high staining of the nails and 5 represents removal with no staining.

TABLE III
Evaluation of Red Nail Enamel

| | | | Composition 3 |
|---|---|---|---|
| | Composition 1 | Composition 2 | (inventive) |
| | (comparative) | (comparative) | [PVA-SbQ + |
| PROPERTY | [latex film former] | [PVA-SbQ] | latex film former] |
| Color Unifor- | 5 | 1 | 5 |
| mity upon | | | |
| Application | | | |
| Wear after 3 | 5 | 1 | 5 |
| hours | | | |
| Wear after 4 | 2 | 0 | 4 |
| days | | | |
| Shine upon | 5 | 3 | 5 |
| Application | | | |
| Shine Reten- | 3 | 0 | 4 |
| tion after | | | |
| 4 days | | | |
| Ease of | 3 | 5 | 5 |
| Removal | | | |

Upon application, comparative composition 2 was observed to have poor color uniformity (poor pigment dispersion) and low shine, while inventive composition 3 and comparative composition 1 exhibited excellent color uniformity and shine. After three hours of wear, comparative composition 2 was observed to have poor adhesion to the nail surface, while inventive composition 3 and comparative composition 1 exhibited excellent adhesion. After four days of wear, comparative composition 1 exhibited poor adhesion and comparative composition 2 exhibited no adhesion to the nail surface, whereas inventive composition 3 continued to exhibit excellent adhesion. In addition, after four days of wear, comparative composition 1 exhibited reduced shine and comparative composition 2 exhibited no shine (no film present), whereas inventive composition 3 exhibited excellent shine retention. Upon removal, comparative composition 1 was observed to stain the nail, whereas compositions comprising PVA-SbQ (comparative composition 2 and inventive composition 3) exhibited minimum staining of the nails.

What is claimed is:

1. A cosmetic composition comprising:
(a) at least one polyvinyl alcohol-styrylpyridinium polymer, and
(b) at least one latex film former.

2. The cosmetic composition of claim 1, wherein the at least one polyvinyl alcohol-styrylpyridinium polymer is chosen from polyvinyl alcohol polymers comprising at least one pendent styrylpyridinium group of formula (I): 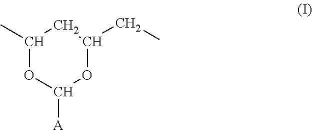
wherein A is chosen from:
(1) groups of formula (II): 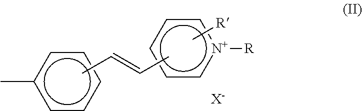
wherein R is chosen from hydrogen, alkyl groups, and hydroxyalkyl groups; R′ is chosen from hydrogen and alkyl groups; and X⁻ is an anion chosen from chloride, bromide, iodide, sulfates, methosulfates, perchlorates, phosphates, and sulfonates; and
(2) groups of formula (III): 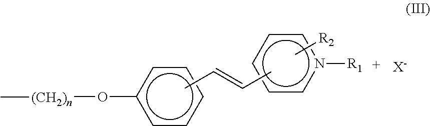
wherein R₁ is chosen from hydrogen, alkyl groups, and aralkyl groups; R₂ is chosen from hydrogen and alkyl groups; X⁻ is an anion chosen from chloride, bromide, iodide, sulfates, methosulfates, perchlorates, phosphates, and sulfonates; and n is an integer ranging from 1 to 6.

3. The cosmetic composition of claim 1, wherein the at least one polyvinyl alcohol-styrylpyridinium polymer is poly(vinyl alcohol), N-methyl-4(4′-formylstyryl)pyridinium methosulfate acetal.

4. The cosmetic composition of claim 1, wherein the ratio of PVA:SbQ in the at least one polyvinyl alcohol-styrylpyridinium polymer ranges from about 99:1 to about 90:10.

5. The cosmetic composition of claim 1, wherein the at least one polyvinyl alcohol-styrylpyridinium polymer is present in the cosmetic composition in an amount ranging from about 0.5% to about 30% by weight.

6. The cosmetic composition of claim 1, wherein the at least one latex film former is chosen from acrylate copolymers, styrene/acrylate copolymers, acrylamide/acrylate copolymer, polyurethanes, and derivatives thereof.

7. The cosmetic composition of claim 6, wherein the at least one latex film former is chosen from ammonium acrylates copolymer, styrene/acrylates copolymer, and styrene/acrylates/ammonium methacrylates copolymer.

8. The cosmetic composition of claim 1, wherein the at least one latex film former is present in the cosmetic composition in an amount ranging from about 20% to about 80% by weight.

9. The cosmetic composition of claim 1, chosen from nail compositions, make-up compositions, mascara compositions, hair-care compositions, and sunscreen compositions.

10. The cosmetic composition of claim 1, chosen from nail compositions.

11. A method of making up and/or enhancing the appearance of a keratinous substrate comprising:
(1) forming a film on the keratinous substrate by applying to said keratinous substrate a cosmetic composition comprising:
(a) at least one polyvinyl alcohol-styrylpyridinium polymer, and
(b) at least one latex film former, and
(2) exposing the film to radiation chosen from UV and visible light radiation.

12. The method of claim 11, wherein the at least one polyvinyl alcohol-styrylpyridinium polymer is chosen from polyvinyl alcohol polymers comprising at least one pendent styrylpyridinium group of formula (I): 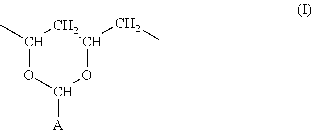
wherein A is chosen from:
(1) groups of formula (II): 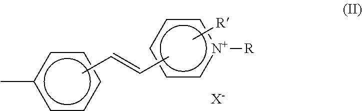
wherein R is chosen from hydrogen, alkyl groups, and hydroxyalkyl groups; R′ is chosen from hydrogen and alkyl groups; and X⁻ is an anion chosen from chloride, bromide, iodide, sulfates, methosulfates, perchlorates, phosphates, and sulfonates; and
(2) groups of formula (III): 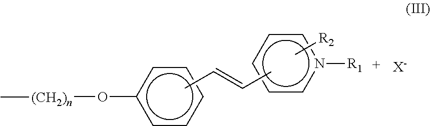
wherein R₁ is chosen from hydrogen, alkyl groups, and aralkyl groups; R₂ is chosen from hydrogen and alkyl groups; X⁻ is an anion chosen from chloride, bromide, iodide, sulfates, methosulfates, perchlorates, phosphates, and sulfonates; and n is an integer ranging from 1 to 6.

13. The method of claim 11, wherein the at least one polyvinyl alcohol-styrylpyridinium polymer is poly(vinyl alcohol), N-methyl-4(4′-formylstyryl)pyridinium methosulfate acetal.

14. The method of claim 11, wherein the ratio of PVA:SbQ in the at least one polyvinyl alcohol-styrylpyridinium ranges from about 99:1 to about 90:10.

15. The method of claim 11, wherein the at least one polyvinyl alcohol-styrylpyridinium is present in the cosmetic composition in an amount ranging from about 0.5% to about 30% by weight.

16. The method of claim 11, wherein the at least one latex film former is chosen from acrylate copolymers, styrene/acrylate copolymers, acrylamide/acrylate copolymer, polyurethanes, and derivatives thereof.

17. The method of claim 16, wherein the at least one latex film former is chosen from ammonium acrylates copolymer, styrene/acrylates copolymer, and styrene/acrylates/ammonium methacrylates copolymer.

18. The method of claim 11, wherein the at least one latex film former is present in the cosmetic composition in an amount ranging from about 20% to about 80% by weight.

19. The method of claim 11, wherein the cosmetic composition is chosen from nail compositions, make-up compositions, mascara compositions, hair-care compositions, and sunscreen compositions.

20. The method of claim 11, wherein the film is exposed to radiation having a wavelength ranging from about 200 nm to about 800 nm.

21. The method of claim 11, wherein the film is exposed to radiation for a time period ranging from about 1 minute to about 30 minutes.
